(19) United States
(12) Patent Application Publication
(10) Pub. No.: US 2005/0075579 A1
(43) Pub. Date: Apr. 7, 2005

(54) Diagnosis of disease by determination of electrical network properties of a body part

(76) Inventors: Adam Semlyen, Toronto (CA); Milan Graovac, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/809,473

(22) Filed: Mar. 26, 2004

Related U.S. Application Data
(63) Continuation-in-part of application No. 10/397,327, filed on Mar. 27, 2003, now abandoned.

(51) Int. Cl. A61B 5/05

(52) U.S. Cl. 600/547

(57) ABSTRACT

A system and method for detecting and diagnosing disease states in a body part is described. The method is based on a set of electrical impedance measurements taken on the surface of a body part and a representation of the body part in the form of a network of impedances that would result in the same surface measurements as the actual body part. The system includes an electrical data unit for measuring electrical data of the body part, the electrical data unit having a plurality of Nₑ electrodes. The system also includes a network module for representing the body part by a network, the network having external nodes and internal nodes connected by current pathways. The system further includes an electrical properties module for determining electrical properties of the pathways using the measured electrical data, and a diagnosis module for utilizing the electrical properties to diagnose the possibility of disease in the body part. It is well known, for example, that tissue electrical impedance changes with malignancy.

### CROSS REFERENCE TO RELATED APPLICATIONS

This application is a continuation-in-part of application Ser. No. 10/397,327, filed on Mar. 27, 2003, the contents of which are incorporated herein by reference.

### FIELD OF THE INVENTION

This invention relates to a method for detecting and diagnosing disease states in body parts of living organisms by using a plurality of electrical impedance measurements.

### BACKGROUND OF THE INVENTION

Measurement devices of various kinds have been used to diagnose disease. For example, x-ray machines measure tissue density, ultrasound machines measure acoustic density, and thermal sensors measure differences in tissue heat generation and conduction; all such measurements can have diagnostic value. In addition to these devices, there exist devices that can measure electrical data, such as voltage between two points when a unit current is injected between other two points. If each point of the current injection is in immediate proximity of one of the voltage measurement points the measured value is equal to the impedance of the body part between the current injecting electrodes.

Values of electrical impedance of various types of body tissue are well known through in vivo studies on humans or from excised tissue made available following therapeutic surgical procedures. It is well documented that a decrease in electrical impedance occurs in the tissue as it undergoes cancerous changes. This finding is consistent over many animal species and tissue types, including, for example, human breast cancers.

One technique for screening and diagnosing diseased states within the body using electrical impedance is disclosed in U.S. Pat. No. 6,122,544, which is incorporated herein by reference. In this patent, data are obtained from two anatomically homologous body regions, one of which may be affected by disease. One subset of the data so obtained is processed and analyzed by structuring the data values as elements of an impedance matrix. Such matrices can be further characterized by their eigenvalues and eigenvectors. These matrices and/or their eigenvalues and eigenvectors can be subjected to a pattern recognition process to match for known normal or abnormal (resulting from disease) matrix or eigenvalue and eigenvector patterns. The matrices and/or their eigenvalues and eigenvectors derived from each homologous body region can also be compared, respectively, to each other using various analytical methods and then subjected to criteria established for differentiating normal from diseased states.

The published international patent application, PCT/CA01/01788, discloses a breast electrode array for diagnosing the presence of a disease state in a living organism, wherein the electrode array comprises a flexible body, a plurality of flexible arms extending from the body, and a plurality of electrodes provided by the flexible arms, where the electrodes are arranged on the arms to obtain impedance measurements between respective electrodes. In one embodiment, the plurality of flexible arms are spaced around the flexible body and provided with an electrode pair. In operation, the electrodes are selected so that the impedance data obtained will include elements of an n×n impedance matrix, plus other impedance values that are typically obtained with tetrapolar impedance measurements. In a preferred embodiment the differences between corresponding homologous impedance measurements in the two body parts are compared in a variety of ways that allow the calculation of metrics that can serve either as an indicator of the presence of disease or localize the disease to a specific breast quadrant or sector. The impedance differences are also displayed graphically, for example in a frontal plane representation of the breast by partitioning the impedance differences into pixel elements throughout the plane.

However convenient this method is, obtaining impedance differences in this manner is simplistic. When current is injected, the flow of electricity through the body part is expected to take one or more complex, winding paths from the starting electrode to the final electrode. The conventional approach does not account for the internal current pathways that the electricity follows.

### SUMMARY OF THE INVENTION

The purpose of the procedure to be described is to obtain a representation of a part of the human body in the form of an electric network or some equivalent to it. The usefulness of such a representation is due to the fact that physiological properties, such as alterations of structure due to a tumor, are generally associated with changes of electric conductivity. Therefore the resultant representation has diagnostic value for detection of anomalies.

In particular, a system for diagnosing the possibility of disease in a body part is described. The system includes a data acquisition module for acquiring the impedance matrix of the body part, as disclosed in U.S. Pat. No. 6,122,544. The system further includes a network module for representing the body part by an electric network of branches with initially unknown branch impedances, the network having external nodes corresponding to the location of the electrodes from the data acquisition module and internal nodes that cannot be accessed from the outside of the body part. The internal and external nodes are connected by current pathways. The system also includes an electrical properties module for determining electrical properties of the pathways using the measured electrical data, and a diagnosis module for utilizing the electrical properties to diagnose the possibility of disease in the body part.

The electrical properties module uses measured data and numerical techniques to determine the admittance (impedance) of each of the current pathways, thereby obtaining an admittance matrix for the particular network representation of the body part. The electrical properties module can likewise repeat these steps to obtain the admittance matrix associated with a homologous body part. For instance, the admittance matrices associated with the left and the right breast can be obtained and then compared by the diagnosis module to diagnose disease.

Also described herein is a method for diagnosing the possibility of disease in a body part. The method includes measuring electrical data of the body part with a set of Nₑ electrodes, and representing the body part by an electric network. The network has external nodes corresponding to the location of the electrodes and internal nodes, the internal and external nodes being connected by current pathways. The method also includes determining electrical properties of the pathways using the measured electrical data, and utilizing the electrical properties to diagnose the possibility of disease in the body part.

### BRIEF DESCRIPTION OF THE DRAWINGS

FIG. 1 is a data flow diagram of the method for detecting and diagnosing the possibility of disease in a body part;

FIG. 2 is a sample network of nodes and current pathways, according to one embodiment of the present invention;

FIG. 3 is a data flow diagram of the electrical properties module of FIG. 1;

FIG. 4 is a data flow diagram of the diagnosis module of the diagnostic system of FIG. 1;

FIG. 5 is a flow chart of the algorithm illustrating the method performed by the diagnostic system of FIG. 1 to diagnose disease; and

FIG. 6 is a Current Pathway Difference (CPD) Plot for an actual subject in one embodiment of the present invention.

### DETAILED DESCRIPTION OF THE INVENTION

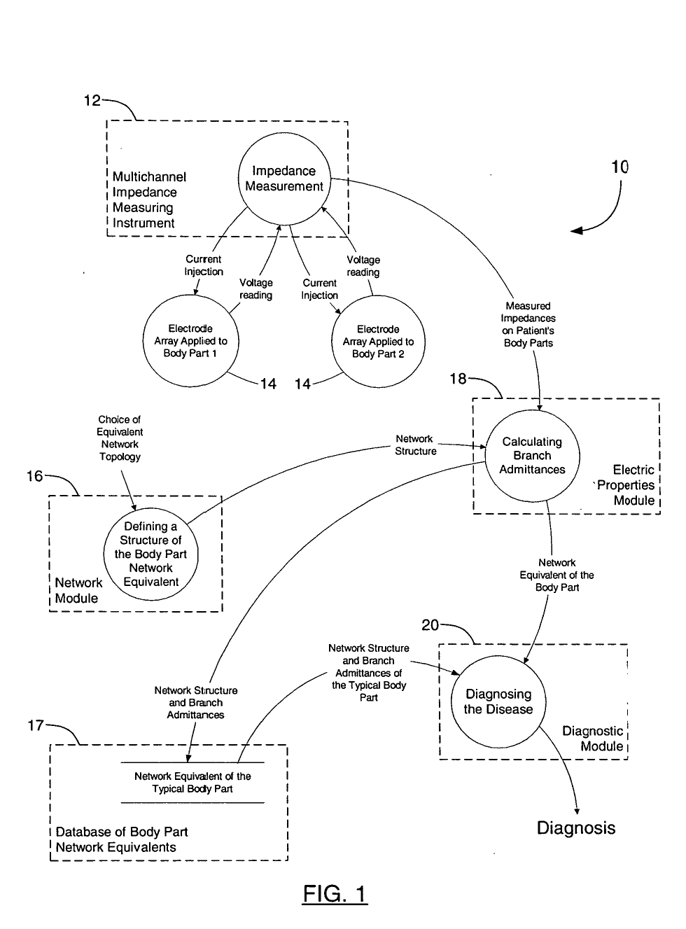

FIG. 1 shows the outline of the proposed method for detecting and diagnosing disease in a body part, such as cancer in a breast. The method uses impedance measurements taken from the multi-channel impedance-measuring instrument 12 with the pair of electrode arrays 14 similar to the one described in PCT/CA01/01788, a network module 16, an electric properties module 18 and a diagnostic module 20. In one embodiment, the electrode array 14 includes Nₑ current injection electrodes, and Nₑ voltage measurement electrodes that are applied on the body part, each of the current injection electrodes being associated with the adjacent voltage measurement electrode. Two sets of measurements are performed. In the first, the impedance is measured between two voltage electrodes when the current is injected between associated current electrodes. The total number of independent current injections and related impedances in the first set is N_{cI}=Nₑ·(Nₑ−1)/2. In the second set of measurements the complete impedance matrix with respect to a base electrode N_{B} is obtained, where N_{B} can be taken to be No for example. For each of Nₑ−1 current injections, iₖ, applied between current electrodes k and N_{B}, where k=1, 2, . . . , Nₑ, k≠N_{B},voltages Vₖⱼ between each of the voltage electrodes j=1, 2, . . . , Nₑ and the base N_{B} are measured. In the second set of measurements there are Nₑ−1 current injections and for each current injection there are Nₑ−1 voltage measurements. The voltages thus obtained Vₖⱼ are divided by the injected current iₖ. The resulting impedances can be organized in a so-called Impedance Matrix (IM) having elements zᵢⱼ=VᵢⱼlIⱼ, as described in U.S. Pat. No. 6,122,544.

In the first set of measurements the impedance is measured n_{CI} times resulting in n_{CI} impedance values, {Z₁^{M}, Z₂^{M}, . . . , Zₙ_{_{CI}}^{M}}, where Zⱼ^{M} is the impedance measured between the voltage electrodes associated with there j^{th} current injection electrode pair when current is injected between associated current electrodes, as required in tetrapolar impedance measurement.

From the second set of measurements the impedance matrix is obtained. In one embodiment of the invention where N_{B}=Nₑ the following impedance matrix is obtained:

$${Z}_{{N}_{B}} = [ \begin{matrix}{z}_{11} & {z}_{12} & ⋯ & {z}_{1 ( {N}_{e} - 1 )} \\ {z}_{21} & {z}_{22} & ⋯ & {z}_{2 ( {N}_{e} - 1 )} \\ ⋮ & ⋮ & & ⋮ \\ {z}_{( {N}_{e} - 1 ) 1} & {z}_{( {N}_{e} - 1 ) 2} & ⋯ & {z}_{( {N}_{e} - 1 ) ( {N}_{e} - 1 )}\end{matrix} ] \qquad ( 1 )$$

The choice of a base electrode is arbitrary. Multiple instances of IM for different base electrodes could be used to reduce the effect of measurement errors.

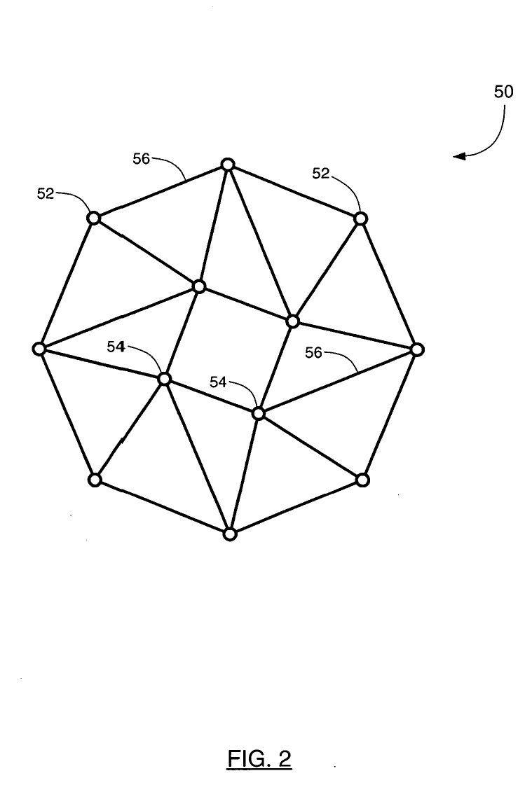

Network module 16 includes hardware and/or software for representing the body part by a network. A number of different network topologies are possible with a given array 14. The topology to be used is an input parameter. The network has external nodes corresponding to the location of electrodes in the electrode array 14 and internal nodes. The external nodes lie on the perimeter of the network, while the internal nodes lie inside. A current pathway is a line segment that connects any two, and only two, nodes. Current pathways intersect only at external nodes or internal nodes, and are the conduits through which current flows. The term “branch” will be used interchangeably with the term “current pathway.” Each branch is associated with two nodes and a branch admittance/impedance. A description of the network appears below in connection with FIG. 2.

The electrical properties module 18 includes software and/or hardware for determining electrical properties of the current pathways using the measured electrical data. For example, the electrical properties module 18 can use the electrical data obtained by the multichannel impedance measuring instrument 12 to find the impedance/admittance of each of the current pathways in the network as specified by the grid module 16. In the preferred embodiment of the invention, the network is represented by a conductance matrix.

Diagnosis module 20 utilizes the determined electrical properties of the pathways to diagnose the possibility of disease in the body part. For example, diagnosis module 20 can compare the conductance matrix of the body part to an average matrix obtained from a population group, or to a conductance matrix obtained from a homologous body part, as described in more detail below.

FIG. 2 shows one possible network topology 50. The network 50 includes eight external nodes 52, four internal nodes 54 and twenty-four current pathways 56. External nodes coincide with the location of the electrodes. All other nodes are called internal nodes. Internal nodes are not directly accessible and observable. The total number of external nodes is the same as the number of the current electrodes Nₑ, while the number of internal nodes depends on a chosen network topology.

The electrical properties module 18 calculates electrical properties, such as the admittances of the pathways 56. The sample network 50 represents the body part and the pathways represent equivalent paths where the current travels when injected into the body part. By using such a network, a more complete description can be obtained of the conductance properties of the body part as compared to a network in which no internal nodes are included. In the example of FIG. 2, the current pathways are line segments that intersect only at external nodes or internal nodes.

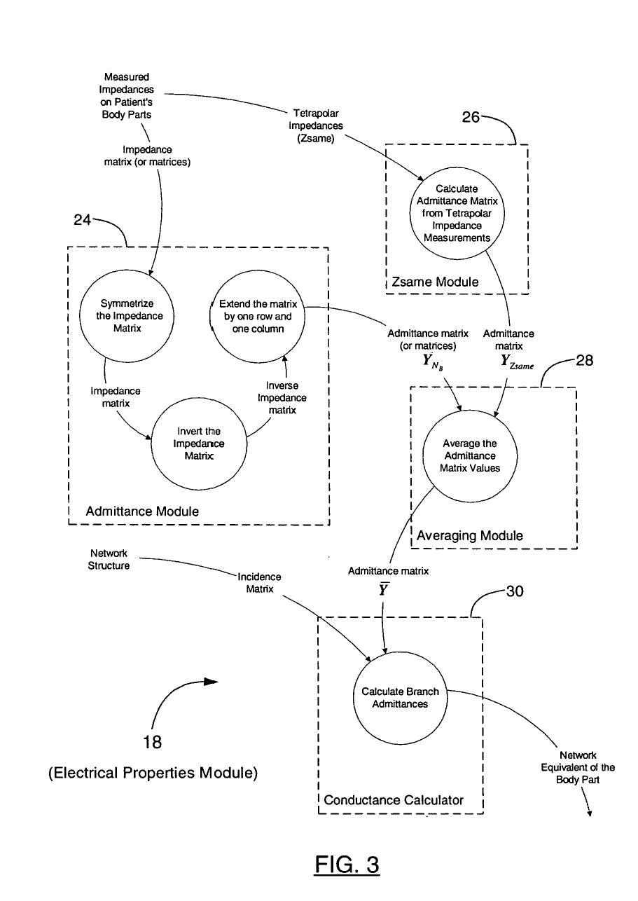

FIG. 3 shows the electrical properties module 18 of FIG. 1. The electrical properties module 18 includes an admittance module 24, Zsame module 26, averaging module 28 and a conductance calculator 30. The admittance module 24 calculates an Nₑ×Nₑ admittance matrix Y_{N}_{_{B}} from the impedance matrix Z_{N}_{_{B}} provided by the multichannel impedance measuring instrument 12 for the base electrode N_{B},as known to those of ordinary skill.

Zsame module 26 calculates the admittance matrix Y_{Zsame} from tetrapolar impedance measurements {Z₁^{M}, Z₂^{M}, . . . , Zₙ_{_{CI}}^{M}} as follows. Zsame is an upper triangular matrix of impedances whose non-zero elements can be represented as a vector, s. As mentioned above, the impedance matrix Z is the matrix obtained using one node as a base node, N_{B}, which may be taken to be the last node., N. Zsame can be obtained from the elements of Z, zᵢⱼ, according to
Zsame_{i,j-1}=Z_{j,i}−2Z_{i,j}+Z_{j,j}
The upper triangular part of the impedance matrix can also be arranged in vector form, similar to the way s was obtained. The relationship between z and s is based on the last equation with indices set in the appropriate fashion for the vector form and is described using the matrix C that satisfies the equation:
s=C*z
The condition number of C is such that C⁻¹ can be derived with virtually no error. In this case, elements of the impedance matrix can be obtained from z=C⁻¹s. The impedance matrix Z may now be obtained from z, making use of the fact that the impedance matrix is symmetric to obtain the lower triangular part. Once the impedance matrix in respect to N-th external node is obtained, the admittance matrix can be easily obtained by inverting it and extending it by one row and column.

The averaging module 28 calculates the average admittance matrix \overline{Y} from admittance matrices Y_{Zsame} and Y_{N}_{_{B}}. In other embodiments, the average can be obtained by averaging Y_{Zsame}, Y_{N}_{_{B}} and other impedance matrices obtained with varying base electrodes. Another possibility is to not average at all, and instead to use just one of the aforementioned impedance matrices.

The conductance calculator 30 calculates the complete (Nₑ+Nᵢ)×(Nₑ+Nᵢ) conductance matrix, G, for the body part by using the average admittance matrix \overline{Y} using Equation (4), as described below. The conductance matrix characterizes the conductance properties of the body part. Because the presence of some diseases, such as cancer, is known to alter the conductance of a body part, the conductance matrix possesses considerable diagnostic value.

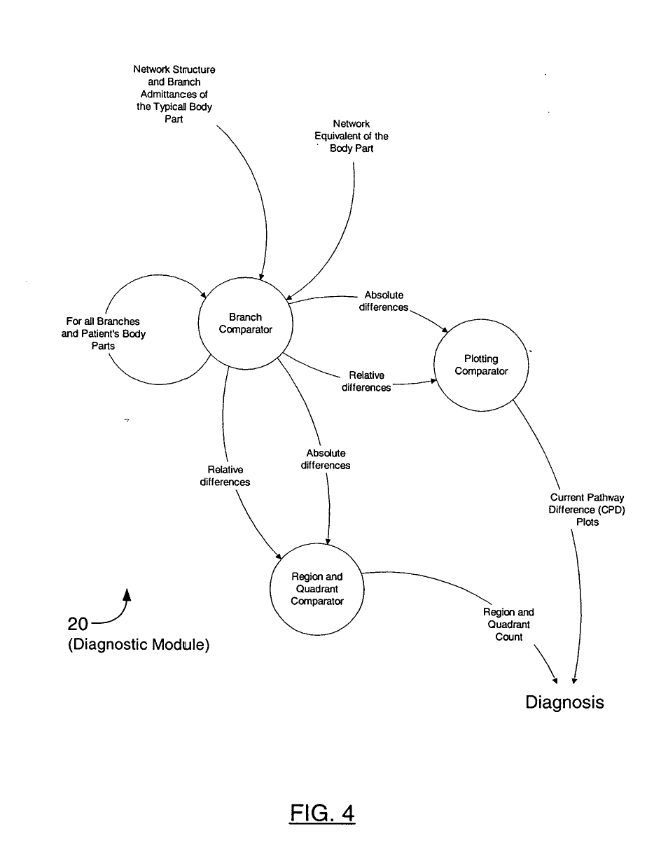

FIG. 4 shows the diagnostic module 20 of FIG. 1. The diagnostic module 20 includes hardware and/or software for comparing the branch conductances of the body part to conductances of corresponding branches of a typical body part obtained from a population group, making use of the fact that off-diagonal elements of the conductance matrix are equal to the respective branch conductances. A difference between corresponding branch conductances could indicate the presence of disease. In one embodiment of the invention, the diagnostic module 20 compares the conductance matrix G to a typical conductance matrix G_{Typical}, such as an average conductance matrix of an appropriate population group. Alternatively, or in addition, the diagnostic module 20 can compare the conductance matrix for the body part, such as the left breast G_{Left}, to a conductance matrix for a homologous body part, the right breast G_{Right}. Again, a difference between the two matrices could indicate the presence of disease.

The diagnostic module 20 can calculate the absolute difference (G_{Left}−G_{Right}) or the relative difference

$$( {G}_{Left} - {G}_{Right} ) / ⌊ \frac{1}{2} \cdot ( {G}_{Left} + {G}_{Right} ) ⌋$$

that is indicative of the possibility of disease in the body part or the homologous body part. Where there is a significant difference, further analysis can be performed to discern which of the homologous pair may be cancerous. For example, as described above, it is known that the electrical properties of cancerous tissue deviate from the norm in a predictable way. Thus, the body part having electrical properties more like those of a cancerous body part can be suspect.

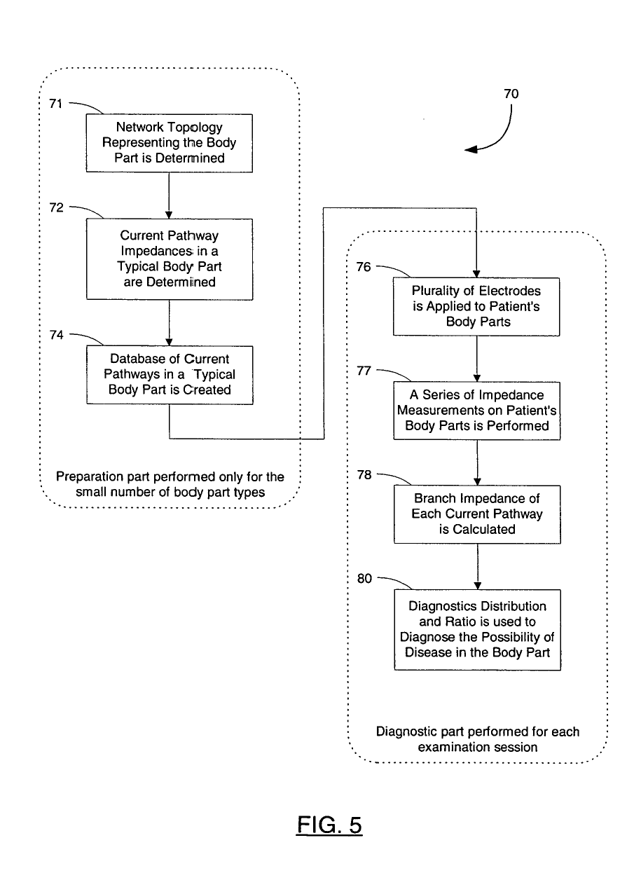

FIG. 5 shows a flowchart that illustrates the main steps 70 utilized by system 10 to diagnose the possibility of disease in a body part. The first part of the procedure is preparatory and involves the acquisition of data for typical, normal body parts as follows: At step (71), the baseline body part is represented with a grid of current pathways. The grid can be two-dimensional, or three-dimensional. Next, at step (72), a number of healthy subjects are analyzed yielding a database (74) of impedances and associated branch admittances for a typical body part. We may repeat these steps to collect several typical sets of data depending on the size, body fat, or some other characteristic of the subject or the body part. This concludes the preparation part. The subject-specific part of the procedure follows: At step (76) a plurality of electrodes is applied to the body part, such as a breast and, at step (77), the electrodes measure the impedance of the body part between electrode pairs. At step (78), the branch impedances of each current pathway are calculated using electrical properties module (18). Subsequently, at step (70), the diagnostic module (20) is utilized to diagnose the possibility of disease in the body part.

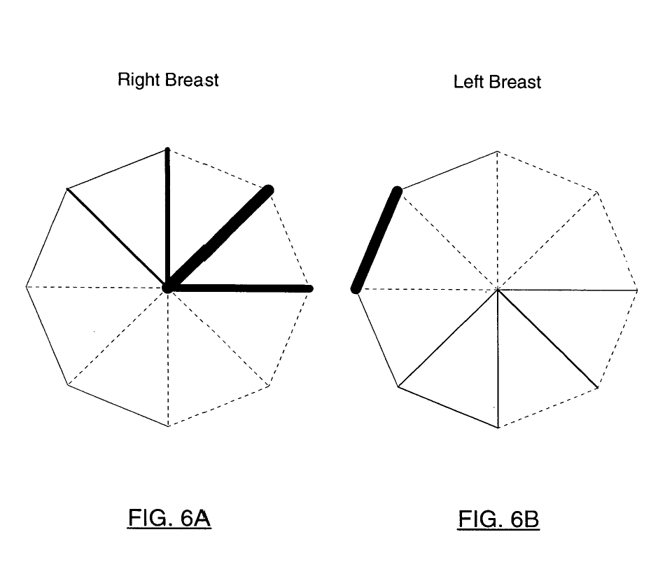

Referring to FIGS. 6A and 6B, sample results in the form of two Current Pathway Difference (CPD) plots are shown illustrating the value of the system and the method of the present invention in detecting breast cancer. In FIGS. 6A and 6B, the right and left breasts, respectively, of a female subject are represented as network plots. To generate these plots, each breast was represented by a network of lines representing current pathways. The branch admittances, as calculated by the diagnostic module 20, are plotted for each of the branches on the left breast element where the conductance of the branch on the left side is higher than the conductance of the analog branch on the right side and on the right breast where the conductance of the branch on the right side is higher than the conductance of the analog branch on the left side, otherwise just dashed lines are shown. The difference is plotted as a line with thickness proportional to the difference (the higher the difference the thicker the line). To obtain the branch admittances, use has been made of the fact known to those of ordinary skill that the branch admittances are non-zero off-diagonal elements of the admittance matrix.

The CPD plot of FIG. 6A indicates the presence of cancer in the upper inner quadrant of the right breast. (Biopsy confirmed a ductal carcinoma in this quadrant.) Observation of FIGS. 6A and 6B shows the preponderance of absolute differences in the diseased region of the right breast (most of the branches with lines), whereas FIG. 6B shows most of its branches as dotted lines.

In one embodiment of the invention the conductance calculator 30 from FIG. 3 obtains the conductance matrix, G, that satisfies:

$$\begin{matrix}G ( \begin{matrix}{v}_{e} \\ {v}_{i}\end{matrix} ) = ( \begin{matrix}{i}_{e} \\ {i}_{i} = 0\end{matrix} ) & ( 2 ) \\ G = ( \begin{matrix}{G}_{ee} & {G}_{ei} \\ {G}_{ei}^{T} & {G}_{ii}\end{matrix} ) . & ( 3 )\end{matrix}$$

where, if Nₑ is the number of external nodes and Nᵢ is the number of internal nodes, then Gₑₑ is an Nₑ×Nₑmatrix, Gₑᵢ is an Nₑ×Nᵢ matrix and Gᵢᵢ is an Nᵢ×Nᵢ matrix. Here, νᵢ is the vector of internal node potentials, νₑ is vector of external node potentials, iₑ is the vector of current injections into external nodes (this does not include the current through branches) and iᵢ is the vector of currents that are injected into internal nodes (these are zero because these nodes are not accessible).

The conductance calculator 30 obtains the conductance matrix by solving the conductance equation
Gₑₑ−GₑᵢGᵢᵢ⁻¹Gₑᵢ^{T}=Gₘ (4)
where Gₘ is the matrix of the real part of the measured admittances Gₘ=Re(\overline{Y}). Equation (4) is obtained from Equation (2) by eliminating the vector of internal potentials, νᵢ, using νᵢ=−Gᵢᵢ⁻¹Gₑᵢ^{T}νₑ, and then substituting \overline{Y}νₑ=iₑ.

The conductance calculator 30 can solve Equation (4) using several methods. In the preferred embodiment of the invention Newton's method for solving a system of nonlinear equations is used. In the general case, there are more equations in (4) than unknowns, thus this set constitutes a linear lease squares problem. This makes it Gauss-Newton. Its solution is a well known iterative procedure.

The only problem may occur when we do not have a good initial guess for conductances. Newton's method is guaranteed to converge only when we are close to the solution. But when we are not, as in the case of a poor initial guess, then what we do is that we bring the “solution” closer to where we are. This may be done by a procedure known as a continuation method. The use of the continuation method assures robustness for the described methodology.

The Gauss-Newton method and a continuation method used in a preferred embodiment of the invention are now described in detail.

### Solution of Conductance Equation

The general form of the conductance equation (Eq. 4) is
f(g)=(Gₑₑ−GₑᵢGᵢᵢ⁻¹Gₑᵢ^{T})−Gₘ=0 (5)
where f(g) is a function of the branch admittances to be arranged in a vector g:

$$\begin{matrix}g = [ \begin{matrix}{g}_{ee} \\ {g}_{ei} \\ {g}_{ie} \\ {g}_{ii}\end{matrix} ] \\ \begin{matrix}{g}_{ee} = [ \begin{matrix}{G}_{ee} ( : , 1 ) \\ ⋮ \\ {G}_{ee} ( : , {N}_{e} )\end{matrix} ] , & {g}_{ie} = [ \begin{matrix}{G}_{ei}^{T} ( : , 1 ) \\ ⋮ \\ {G}_{ei}^{T} ( : , {N}_{e} )\end{matrix} ] ,\end{matrix} \\ \begin{matrix}{g}_{ei} = [ \begin{matrix}{G}_{ei} ( : , 1 ) \\ ⋮ \\ {G}_{ei} ( : , {N}_{e} )\end{matrix} ] , & {g}_{ii} = [ \begin{matrix}{G}_{ii} ( : , 1 ) \\ ⋮ \\ {G}_{ii} ( : , {N}_{i} )\end{matrix} ]\end{matrix}\end{matrix} \qquad ( 6 )$$

(A) Newton's Method for a System of Nonlinear Equations

Let g=[gᵢ], i=1, . . . , N×N, be a column vector of the conductances of all possible pathways for a given N. Let g₀ be a vector of estimated elements of G for the given network. Vector g has zero for each g(i) that corresponds to certain G(p,q) where nodes p and q are not connected. The Jacobian is

$$J ( g ) = \frac{\partial f}{\partial g} .$$

Substituting g₀ in (5), f(g₀)≠0 is calculated.

A new vector g⁽ⁿ⁾ is calculated from:
J(g⁽ⁿ⁾)Δg⁽ⁿ⁾=−f(g⁽ⁿ⁾)
g⁽ⁿ⁺¹⁾=g⁽ⁿ⁾+Δg⁽ⁿ⁾
For g₀ close enough to g* (solution of the problem (5)), the iterative method described by equations (7) converges to g* (quadratically for a full set of equations). If g₀ is not a good initial guess for g*, system (7) may not converge. In that case, the continuation method described below is applied. If the number of equations is greater than the number of unknowns, a least squares problem is being solved using the Gauss-Newton method. In that case, the convergence is not quadratic.
(B) Continuation Method

For a function f:Rⁿ→R^{m}, a numerical method, sensitive to the initial guess, can be used to solve the equation f(g)=0, where g ε Rⁿ. If the guess is not sufficiently close to the solution g*, the numerical method might not converge. To obtain a sufficiently close initial vector g⁽⁰⁾ for problem f(g)=0, an augmented function {double overscore (f)}(g(θ),θ):Rⁿ⁺¹→R^{m} can be considered that is defined as:
{double overscore (f)}(g(θ),θ)=(1−θ)\overline{f}(g(θ))+θf(g(θ)), (8)
and the following equivalent problem solved:
{double overscore (f)}(g(θ),θ)=0, θε[0,1] (9)
where:
\overline{f}(g):Rⁿ→R^{m} is a function of g, such that equation f′(g)=0 is easy to solve, and θ is a real number.
The important properties of the new function are:

$${f}^{\overline{\_}} ( g ( θ ) , θ ) = \{ \begin{matrix}\overline{f} ( g ) , & θ = 0 \\ ( 1 - θ ) \overline{f} ( g ) + θ f ( g ) , & θ ∈ ] 0 , 1 [ \\ f ( g ) , & θ = 1\end{matrix}$$

For θ=0, the equation (9) becomes \overline{f}(g)=0, which is easy to solve by its construction.

For θ=1, the problem (9) is identical to the original problem f(g)=0. For θε]0,1[, the problem (9) becomes
(1−θ)\overline{f}(g(θ))+θf(g(θ))=0 (10)

The following method takes the solution of the problem g=0 as the initial guess g⁽⁰⁾ for the close problem (1−θ)g(θ)+θf(g(θ))=0, where is θ=0+δθ, and δΘ<<1. The underlying assumption is that the solution for the problem {double overscore (f)}(g(θ),θ) is close enough to g*(θ+δθ) for the problem {double overscore (f)}(g(θ+δθ),θ+δθ) so that the numerical algorithm converges to g* (θ+δθ).

To summarize, the continuation method includes a) solving the problem {double overscore (f)}(g(θ),θ)=0 for θ=0, b) taking the solution g*(θ) as the initial guess g⁽⁰⁾(θ+δθ) for
{double overscore (f)}(g(θ+δθ),θ+δθ)=(1−(θ+δθ))\overline{f}(g(θ+δθ))+(θ+δθ)f(g(θ+δθ)), (11)
where δθ is a small step, c) attempting to solve this problem using a Gauss-Newton algorithm, d) if the Gauss-Newton algorithm converges, setting θ=θ+δθ, δθ=α·δθ, where α>1 is an acceleration coefficient, and if it does not converge, setting δθ=δθ/2, and e) repeating step b) until θ=1.

These methods can be used to solve the conductance equation:
f(g)=(Gₑₑ−GₑᵢGᵢᵢ⁻¹Gₑᵢ^{T})−Gₘ (12)
The matrix of finite differences is Δf=[Δf₁ Δf₂ . . . Δf_{Ne}], where
fₖ(g)=(gₑₑₖ−GₑᵢGᵢᵢ⁻¹gᵢₑₖ)−gₘₖ, k=1 . . . Nₑ .
From the above equation,
Δfₖ=Δgₑₑₖ−ΔGₑᵢGᵢᵢ⁻¹gᵢₑₖ−GₑᵢΔGᵢᵢ⁻¹gᵢₑₖ−GₑᵢGᵢᵢ⁻¹Δgᵢₑₖ (13)
Matrix ΔGᵢᵢ⁻¹ can be calculated from the equation Gᵢᵢ⁻¹Gᵢᵢ=I as follows
Gᵢᵢ⁻¹Gᵢᵢ=I|Δ
ΔGᵢᵢ⁻¹Gᵢᵢ+Gᵢᵢ⁻¹ΔGᵢᵢ=0|Gᵢᵢ⁻¹
ΔGᵢᵢ⁻¹=−Gᵢᵢ⁻¹ΔGᵢᵢGᵢᵢ⁻¹ (14)
Substituting (14) in (13) leads to:
Δfₖ=Δgₑₑₖ−ΔGₑᵢ(Gᵢᵢ⁻¹gᵢₑₖ)+(GₑᵢGᵢᵢ⁻¹)ΔGᵢᵢ(Gᵢᵢ⁻¹gᵢₑₖ)−(GₑᵢGᵢᵢ⁻¹)Δgᵢₑₖ (15)
The next step is to reorder the matrix products in (15). Each ΔG_{**} should come as a vector Δg_{**} to the right hand side of the corresponding matrix product. Δg_{**} is a column vector of all columns of ΔG_{**} as indicated by (6). By applying the rule
ΔXB [symbol] AΔx (16)
Equation (16) becomes
Δfₖ=Δgₑₑₖ−M₁ₖΔgₑᵢ+M₂ₖΔgᵢᵢ−M₃Δgᵢₑₖ (17)
where M₃=GₑᵢGᵢᵢ⁻¹, M₂ₖ=GₑᵢGᵢᵢ⁻¹Aₖ, and M₁ₖ and Aₖ are calculated by applying (16):
ΔGₑᵢ(Gᵢᵢ⁻¹gᵢₑₖ)=M₁ₖΔgₑᵢ
ΔGᵢᵢ(Gᵢᵢ⁻¹gᵢₑₖ)=AₖΔgᵢᵢ
Equation (17) can be written as:

$$Δ {f}_{k} = [ \begin{matrix}I & - {M}_{1 k} & {M}_{2 k} & - {M}_{3}\end{matrix} ] \cdot ⌊ \begin{matrix}Δ {g}_{eek} \\ Δ {g}_{ei} \\ Δ {g}_{ii} \\ Δ {g}_{iek}\end{matrix} ⌋ \qquad ( 18 )$$

where Jₖ=[I−M₁ₖM₂ₖM₂ₖ−M₃] is the Jacobian matrix corresponding to the k^{th} column of f(g^{(k)}) in equation (7).

Solving the system

$${J}_{k}^{( n )} \cdot [ \begin{matrix}Δ {g}_{eek}^{( n )} \\ Δ {g}_{ei}^{( n )} \\ Δ {g}_{ii}^{( n )} \\ Δ {g}_{iek}^{( n )}\end{matrix} ] = - {f}_{k}^{( n )} , \qquad k = 1 , … , Ne$$

is equivalent to solving equation (19)

$$⌊ \begin{matrix}I & & & 0 & \vert & - {M}_{11}^{( n )} & \vert & {M}_{21}^{( n )} & \vert & - {M}_{3}^{( n )} & & & 0 \\ & I & & & \vert & - {M}_{12}^{( n )} & \vert & {M}_{22 ( n )} & \vert & & - {M}_{3}^{( n )} & & \\ & & ⋰ & & \vert & ⋮ & \vert & ⋮ & \vert & & & ⋰ & \\ 0 & & & I & \vert & - {M}_{1 Ne}^{( n )} & \vert & {M}_{2 Ne}^{( n )} & \vert & 0 & & & - {M}_{3}^{( n )}\end{matrix} ⌋ \cdot ⌊ \begin{matrix}Δ {g}_{ee}^{( n )} \\ Δ {g}_{ei}^{( n )} \\ Δ {g}_{ii}^{( n )} \\ Δ {g}_{ie}^{( n )}\end{matrix} ⌋ = - ⌊ \begin{matrix}\begin{matrix}\begin{matrix}Δ {f}_{1}^{( n )} \\ Δ {f}_{2}^{( n )}\end{matrix} \\ ⋮\end{matrix} \\ Δ {f}_{Ne}^{( n )}\end{matrix} ⌋ \qquad ( 19 )$$

To simplify equation (19), the vector of admittances may be written as
gg=Tᵣ·g_{branch} (20)
where

$$gg = [ \begin{matrix}{g}_{ee} \\ {g}_{ei} \\ {g}_{ii} \\ {g}_{ie}\end{matrix} ] ,$$

In (20) g_{branch} is a vector of the current pathways in the selected network, while Tᵣ is an incidence matrix defined by the network so that equation (20) holds.

Substituting (20) in (19) results in the reduced system (21):

$${J}_{r}^{( n )} \cdot Δ {g}_{branch}^{( n )} = - [ \begin{matrix}Δ {f}_{1}^{( n )} \\ Δ {f}_{2}^{( n )} \\ ⋮ \\ Δ {f}_{Ne}^{( n )}\end{matrix} ] \qquad ( 21 )$$

where

$${J}_{r} = ⌊ \begin{matrix}I & & & 0 & \vert & - {M}_{11}^{( n )} & \vert & {M}_{21}^{( n )} & \vert & - {M}_{3}^{( n )} & & & 0 \\ & I & & & \vert & - {M}_{12}^{( n )} & \vert & {M}_{22 ( n )} & \vert & & - {M}_{3}^{( n )} & & \\ & & ⋰ & & \vert & ⋮ & \vert & ⋮ & \vert & & & ⋰ & \\ 0 & & & I & \vert & - {M}_{1 Ne}^{( n )} & \vert & {M}_{2 Ne}^{( n )} & \vert & 0 & & & - {M}_{3}^{( n )}\end{matrix} ⌋ \cdot {T}_{r}$$

and Δg_{branch} is a vector of current pathways admittance corrections. The new admittance vector g_{branch}⁽ⁿ⁺¹⁾ is calculated from the equation

$${g}_{branch}^{( n + 1 )} = {g}_{branch}^{( n )} + Δ {g}_{branch}^{( n )} \qquad ( 22 )$$

Similar to (20), vector g⁽ⁿ⁺¹⁾ can be calculated from

$${g}_{branch}^{( n + 1 )}$$

as

$${g}^{( n + 1 )} = T \cdot {g}_{branch}^{( n + 1 )}$$

where T is a matrix defined by the chosen network so that the equation (23) holds.

There are other methods, besides the method just described, which can be used by the conductance calculator 30 for obtaining the branch conductances and subsequently the conductance matrix.

Once the conductance matrix calculator 30 finds the conductance matrix for the body part, using any of the methods described above, the aforementioned steps can be repeated to obtain the conductance matrix of the homologous body part. The diagnosis module 20 can then compare the conductance matrix for the body part and the conductance matrix for the homologous body part by using several comparison methods. For example, the norm of the difference of these two matrices can be computed, and if it is greater than some threshold, then further analysis can be performed as this difference may signal the presence of disease.

Several computer systems can be used to implement the method for diagnosing disease in a body part. The computer system can include a monitor for displaying parts or the whole conductance matrix, or for displaying the difference between the conductance matrix for the body part and the conductance matrix for the homologous body part using one of several visual methods. In one embodiment, the method can be implemented on a 2 GHz Pentium™ 4 system with 512 MB RAM.

It should be understood that various modifications could be made to the embodiments described and illustrated herein, without departing from the present invention, the scope of which is defined in the appended claims. For example, although emphasis has been placed on describing a system for diagnosing breast cancer, the principles of the present invention can also be advantageously applied to other diseases of other body parts.

Additionally, in the above analysis, emphasis was placed on the real conductance matrix G, which was obtained by solving the conductance equation (4); however, it should be understood that an analogous analysis in the complex domain could also be carried out by obtaining instead the complex admittance matrix Y, given by

$$Y = ( \begin{matrix}{Y}_{ee} & {Y}_{ei} \\ {Y}_{ei}^{T} & {Y}_{ii}\end{matrix} ) ,$$

by solving the admittance equation Yₑₑ−YₑᵢYᵢᵢ⁻¹Yₑᵢ^{T}=\overline{Y}, where \overline{Y} is the average of Y_{N}_{_{B}} and Y_{Zsame}. The complex admittance matrix may then be used to diagnose disease in a manner analogous to the way its real part was used above. In the last equation, the average impedance matrix need not be used; instead, one of the aforementioned impedance matrices, Y_{N}_{_{B}} or Y_{Zsame} can be used as the right hand side of this last equation. In this embodiment, where Y instead of G is computed, analogous steps are taken to those described above, mutatis mutandis (for example, replacing the conductance calculator 30 by an impedance calculator 30).

1. A method for diagnosing the possibility of disease in a body part, the method comprising measuring electrical data of the body part with a plurality of Nₑ electrodes;
representing the body part by a network, said network having external nodes corresponding to the location of the plurality of the Nₑ electrodes and internal nodes, wherein the internal and external nodes are connected by current pathways;
determining electrical properties associated with the pathways using the measured electrical data; and
utilizing the electrical properties to diagnose the possibility of disease in the body part.

2. The method of claim 1, wherein the step of measuring includes measuring an impedance zᵢⱼ of the body part.

3. The method of claim 2, wherein the step of measuring an impedance zᵢⱼ of the body part includes
injecting a current iᵢbetween an i^{th} electrode chosen from among the plurality and a base electrode taken to be the Nₑ^{th} electrode of the plurality;
measuring a resultant voltage Vᵢⱼ between a j^{th} electrode chosen from among the plurality and the base electrode; and
repeating the steps of injecting and measuring to calculate an Nₑ−1×Nₑ−1 impedance matrix Z_{N}_{_{B}} with matrix elements zₘₙ given by zₘₙ=Vₘₙ/iₘ where 1≦i≦Nₑ−1 and 1≦j≦Nₑ−1.

4. The method of claim 3, wherein the step of determining electrical properties associated with the pathways includes calculating an Nₑ×N, admittance matrix Y_{N}_{_{B}} from the impedance matrix Z_{N}_{_{B}}.

5. The method of claim 4, wherein the step of determining electrical properties associated with the pathways further includes determining a conductance matrix, G, for the body part by using the admittance matrix Y_{N}_{_{B}}.

6. The method of claim 5, wherein the step of utilizing the electrical properties includes
obtaining a typical conductance matrix from a population group; and
comparing the conductance matrix for the body part and the typical conductance matrix from a population group to diagnose the possibility of disease in the body part.

7. The method of claim 5, wherein the step of utilizing the electrical properties includes
determining a conductance matrix for a homologous body part; and
comparing the conductance matrix for the body part and the conductance matrix for the homologous body part to diagnose the possibility of disease in the body part.

8. The method of claim 7, wherein the step of comparing includes displaying a first representation of the body part having current pathways with associated branch conductances and a second representation of the body part having current pathways with associated branch conductances.

9. The method of claim 5, wherein the step of determining a conductance matrix includes solving the equation
Gₑₑ−GₑᵢGᵢᵢ⁻¹Gₑᵢ^{T}=Re(Y_{N}_{_{B}})
where, if Nₑ is the number of external nodes and Nᵢ is the number of internal nodes, then Gₑₑ is an Nₑ×Nₑ matrix, Gₑᵢ is an Nₑ×Nᵢ matrix and Gᵢᵢ is an Nᵢ×Nᵢ matrix defined by
$$G = ( \begin{matrix}{G}_{ee} & {G}_{ei} \\ {G}_{ei}^{T} & {G}_{ii}\end{matrix} ) .$$

10. The method of claim 9, wherein solving the equation includes utilizing at least one of Newton's method for a system of nonlinear equations and a continuation method.

11. The method of claim 5, wherein the step of measuring electrical data further includes calculating an average impedance matrix \overline{Y}, by averaging Y_{N}_{_{B}} and a second admittance matrix Y_{Zsame}.

12. The method of claim 11, wherein the step of determining a conductance matrix includes solving the equation
Gₑₑ−GₑᵢGᵢᵢ⁻¹Gₑᵢ^{T}=Re(\overline{Y}),
where, if Nₑ is the number of external nodes and Nᵢ is the number of internal nodes, then Gₑₑ is an Nₑ×N, matrix, Gₑᵢ is an Nₑ×Nᵢ matrix and Gᵢᵢ is an Nₑ×Nᵢ matrix defined by
$$G = ( \begin{matrix}{G}_{ee} & {G}_{ei} \\ {G}_{ei}^{T} & {G}_{ii}\end{matrix} ) .$$

13. The method of claim 12, wherein solving the equation includes utilizing at least one of Newton's method for a system of nonlinear equations and a continuation method.

14. The method of claim 1, wherein the current pathways intersect only at external nodes or internal nodes.

15. The method of claim 1, wherein the current pathways are line segments of the network.

16. The method of claim 1, wherein the external nodes lie on a perimeter of the network and the internal nodes lie inside the perimeter.

17. The method of claim 1, wherein the step of determining electrical properties associated with the pathways includes determining a conductance matrix, G, for the body part by using at least one of an admittance matrix Y_{N}_{_{B}} obtained from impedance measurements of the body part and an admittance matrix Y_{Zsame} obtained from tetrapolar impedance measurements of the body part.

18. The method of claim 1, wherein the electrical properties are elements of an admittance matrix Y, the step of determining including obtaining the admittance matrix by solving an admittance equation.

19. A system for diagnosing the possibility of disease in a body part, the system comprising
an electrode array for measuring electrical data of the body part, said electrode array having a plurality of Nₑ electrodes;
a network module for representing the body part by a network, said network having external nodes corresponding to the location of the plurality of the Nₑ electrodes and internal nodes, wherein the internal and external nodes are connected by current pathways;
an electrical properties module for determining electrical properties associated with the pathways using the measured electrical data; and
a diagnosis module for utilizing the electrical properties to diagnose the possibility of disease in the body part.

20. The system of claim 19, further comprising a multichannel impedance measuring instrument for obtaining an impedance Zᵢⱼ of the body part.

21. The system of claim 20, wherein, after the electrode array is used to a) inject a current iᵢ between an i^{th} electrode chosen from among the plurality and a base electrode taken to be the Nₑ^{th} electrode of the plurality, and b) measure a resultant voltage Vᵢⱼ between a j^{th} electrode chosen from among the plurality and the reference electrode, a multichannel impedance measuring instrument calculates zᵢⱼ according to zᵢⱼ=Vᵢⱼ/iᵢ where 1≦i≦Nₑ−1 and 1≦i≦Nₑ−1.

22. The system of claim 21, wherein, after steps a) and b) are repeated, the impedance module calculates an Nₑ−1×Nₑ−1 impedance matrix Z_{N}_{_{B}} with matrix elements zₘₙ given by zₘₙ=Vₘₙ/iₘ where 1≦i≦Nₑ−1 and 1≦j≦Nₑ−1.

23. The system of claim 22, wherein the electrical properties module includes an admittance module for calculating an Nₑ×Nₑ admittance matrix Y_{N}_{_{B}} from the impedance matrix Z_{N}_{_{B}}.

24. The system of claim 23 wherein the electrical properties module further includes a conductance calculator for determining a conductance matrix, G, for the body part by using the admittance matrix.

25. The system of claim 24, wherein the diagnosis module compares the conductance matrix for the body part to a typical conductance matrix from a population group to diagnose the possibility of disease in the body part.

26. The system of claim 24, wherein the diagnosis module compares the conductance matrix for the body part to a conductance matrix for a homologous body part to diagnose the possibility of disease in the body part.

27. The system of claim 24, wherein the conductance calculator solves the equation
Gₑₑ−GₑᵢGᵢᵢ⁻¹Gₑᵢ^{T}=Re(Y_{N}_{_{B}}),
where, if Nₑ is the number of external nodes and Nᵢ is the number of internal nodes, then Gₑₑ is an Nₑ×Nₑ matrix, Gₑᵢ is an Nₑ×Nᵢ matrix and Gᵢᵢ is an Nᵢ×Nᵢ matrix defined by
$$G = ( \begin{matrix}{G}_{ee} & {G}_{ei} \\ {G}_{ei}^{T} & {G}_{ii}\end{matrix} ) .$$

28. The system of claim 27, wherein the conductance calculator solves the equation
Gₑₑ−GₑᵢGᵢᵢ⁻¹Gₑᵢ^{T}=Re(Y_{N}_{_{B}})
by utilizing at least one of Newton's method for a system of nonlinear equations and a continuation method.

29. The system of claim 24, wherein the electrical properties module further includes an averaging module for calculating an average impedance matrix \overline{Y}, by averaging Y_{N}_{_{B}} with a second admittance matrix.

30. The system of claim 29, wherein the conductance calculator solves the equation
Gₑₑ−GₑᵢGᵢᵢ⁻¹Gₑᵢ^{T}=Re(\overline{Y}),
where, if Nₑ is the number of external nodes and Nᵢ is the number of internal nodes, then Gₑₑ is an Nₑ×Nₑ matrix, Gₑᵢ is an Nₑ×Nᵢ matrix and Gᵢᵢ is an Nᵢ×Nᵢ matrix defined by
$$G = ( \begin{matrix}{G}_{ee} & {G}_{ei} \\ {G}_{ei}^{T} & {G}_{ii}\end{matrix} ) .$$

31. The system of claim 30, wherein the conductance calculator solves the equation
Gₑₑ−GₑᵢGᵢᵢ⁻¹Gₑᵢ^{T}=Re(\overline{Y})
by utilizing at least one of Newton's method for a system of nonlinear equations and a continuation method.

32. The system of claim 19, wherein the current pathways intersect only at external nodes or internal nodes.

33. The system of claim 19, wherein the current pathways are line segments of the network.

34. The system of claim 19, wherein the external nodes lie on a perimeter of the network and the internal nodes lie inside the perimeter.

35. The system of claim 19, further comprising at least one of an admittance module for obtaining an admittance matrix Y_{N}_{_{B}} from impedance measurements of the body part and a Zsame module for obtaining an admittance matrix Y_{Zsame} from tetrapolar impedance measurements of the body part, the at least one the admittance matrix Y_{N}_{_{B}} and the admittance matrix Y_{Zsame} used by the electrical properties module to obtain elements of a conductance matrix G as the electrical properties.

36. The system of claim 19, wherein the electrical properties are elements of an admittance matrix Y, the electrical properties module including an impedance calculator for obtaining the admittance matrix by solving an admittance equation.
